(19) United States
(12) Patent Application Publication
(10) Pub. No.: US 2007/0196487 A1
(43) Pub. Date: Aug. 23, 2007

(54) METHOD AND APPARATUS FOR DRILLING ORIFICES IN OSMOTIC TABLETS INCORPORATING NEAR-INFRARED SPECTROSCOPY

(76) Inventor: Johan H. Geerke, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/675,559

(22) Filed: Feb. 15, 2007

Related U.S. Application Data
(60) Provisional application No. 60/774,013, filed on Feb. 16, 2006.

(51) Int. Cl. A61K 9/24; B23B 39/04

(52) U.S. Cl. 424/473; 408/8

(57) ABSTRACT

The present invention relates to apparatus and methods for the manufacture of osmotic tablets, wherein near-infrared spectroscopy systems are used.

### FIELD OF THE INVENTION

The present invention relates to the manufacture of osmotic tablets, in particular to the forming of orifices in osmotic tablets, and to related methods.

### BACKGROUND

Osmotic tablets in general utilize osmotic pressure to generate a driving force for imbibing fluid into a compartment formed, at least in part, by a semipermeable membrane that permits free diffusion of fluid but not drug or osmotic agent(s), if present. A significant advantage to osmotic systems is that operation is pH-independent and thus continues at the osmotically determined rate throughout an extended time period even as the osmotic tablet transits the gastrointestinal tract and encounters differing microenvironments having significantly different pH values. A review of such osmotic tablets is found in Santus and Baker, “Osmotic drug delivery: a review of the patent literature,” Journal of Controlled Release 35 (1995) 1-21. U.S. Pat. Nos. 3,845,770; 3,916,899; 3,995,631; 4,008,719; 4,111,202; 4,160,020; 4,327,725; 4,578,075; 4,681,583; 5,019,397; and 5,156,850 disclose osmotic tablets for the continuous dispensing of active agent.

The present invention is particularly concerned with osmotic tablets in which a drug composition is delivered as a slurry, suspension or solution from an orifice at least in part by the action of an expandable (“push”) layer. Such osmotic tablets are disclosed, among other places, in U.S. Pat. Nos. 5,633,011; 5,190,765; 5,252,338; 5,620,705; 4,931,285; 5,006,346; 5,024,842; and 5,160,743.

Such orifices are often prepared by drilling, either by mechanical drilling or by laser drilling. One problem in the drilling of orifices is knowing where to drill. The orifice is preferably drilled in the drug layer end of the osmotic tablet so that drug can be released appropriately. Drilling the orifice elsewhere, in the push layer end for example, would lead to the malfunction of the osmotic tablet.

Accordingly, the tablet orientation needs to be known in order for the orifice to be drilled in the correct location. This is a complicated problem, because by the time that the orifice is to be drilled, the osmotic tablet has been coated with one or more coatings (such as a semi-permeable membrane), making it very difficult to distinguish between a push layer end and a drug layer end of an osmotic tablet.

Accordingly, methods and apparatus are needed that facilitate the drilling of orifices in the drug layer end of osmotic tablets, at high speeds and with low levels of error.

### SUMMARY OF THE INVENTION

In an aspect, the invention relates to an apparatus comprising: an osmotic tablet handling system for handling osmotic tablets that comprise a drug layer and a push layer; an osmotic tablet laser drilling system for drilling at least one orifice in a drug layer end of the osmotic tablet; a near-infrared spectroscopy system; and a laser drill control system; wherein the osmotic tablet laser drilling system is coupled to the osmotic tablet handling system and the near-infrared spectroscopy system, and the near-infrared spectroscopy system is coupled to the osmotic tablet handling system and a laser drill control system, and the laser drill control system is coupled to the near-infrared spectroscopy system and the osmotic tablet laser drilling system.

In another aspect, the invention relates to a method comprising: handling an osmotic tablet that comprises a drug layer and a push layer; detecting differences between near-infrared spectroscopic characteristics of the drug layer and the push layer of the osmotic tablet being handled; and causing an osmotic tablet laser drilling system to laser drill at least one orifice in a drug layer end of the osmotic tablet, based on the differences detected between near-infrared spectroscopic characteristics of the drug layer and the push layer of the osmotic tablet.

In yet another aspect, the invention relates to an apparatus comprising: means for handling an osmotic tablet that comprises a drug layer and a push layer; means for detecting differences between near-infrared spectroscopic characteristics of the drug layer and the push layer of the osmotic tablet being handled; and means for causing an osmotic tablet laser drilling system to laser drill at least one orifice in a drug layer end of the osmotic tablet, based on the differences detected between near-infrared spectroscopic characteristics of the drug layer and the push layer of the osmotic tablet.

### BRIEF DESCRIPTION OF THE DRAWINGS

FIG. 1 shows infrared spectra of a solid drug layer and push layer of an osmotic tablet.

FIG. 2 shows a near-infrared spectrum obtained for a push layer of an osmotic tablet.

FIG. 3 shows a near-infrared spectrum obtained for a liquid drug layer of an osmotic tablet.

FIG. 4 shows an embodiment of the present invention.

FIG. 5 shows an embodiment of the present invention.

### DETAILED DESCRIPTION

I. Introduction

The inventor has found unexpectedly that it is possible to solve the problems noted above with respect to drilling orifices in the drug layer end of osmotic tablets by an apparatus comprising: an osmotic tablet handling system for handling osmotic tablets that comprise a drug layer and a push layer; an osmotic tablet laser drilling system, coupled to the osmotic tablet handling system, for drilling at least one orifice in a drug layer end of the osmotic tablet; a near-infrared spectroscopy system, coupled to the osmotic tablet handling system; and a laser drill control system, coupled to the near-infrared spectroscopy system and the osmotic tablet laser drilling system. Associated method embodiments of the present invention may also address the problems noted above.

As discussed further below, near-infrared spectroscopy can be used to detect differences between near-infrared spectroscopic characteristics of the drug layer and the push layer of an osmotic tablet being handled by the osmotic tablet handling system. This information can then be fed to a laser drill control system that can then operate to drill at least one orifice in an osmotic tablet present to the osmotic tablet laser drilling system by the osmotic tablet handling system. This system provides for the drilling of orifices in the drug layer end of osmotic tablets, at high speeds and with low levels of error.

The invention will now be described in more detail below.

II. Definitions

All percentages are weight percent unless otherwise noted.

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Osmotic tablet handling system” means an apparatus for handling osmotic tablets in the course of manufacturing the osmotic tablets.

“Osmotic tablets” means pharmaceutical dosage forms that are designed to operate according to osmotic principles. Examples of such osmotic tablets are provided below. In embodiments, osmotic tablets comprise a drug layer and a push layer; such embodiments are described in more detail below. Osmotic tablets according to the invention may be coated; in certain embodiments the coating(s) may be clear, translucent or opaque. In a preferred embodiment, the coatings are opaque.

“Osmotic tablet laser drilling system” means a laser drilling system that operates to drill at least one orifice in an osmotic tablet, preferably at the drug layer end of the osmotic tablet. Such osmotic tablet laser drilling systems are typically coupled, preferably mechanically and/or electronically, to an osmotic tablet handling system. Such coupling facilitates the drilling at least one orifice in a drug layer end of the osmotic tablet.

“Orifice” means a hole or passageway formed through the semi-permeable membrane of an osmotic tablet. Examples of exit ports and methods of making them that are useful in the practice of this invention are presented elsewhere herein.

“Push layer” means a displacement composition that is positioned within the osmotic tablet such that as the push layer expands during use, the materials forming the drug layer are expelled from the osmotic tablet via the at least one orifice located in the semi-permeable membrane.

“Drug layer” means that portion or those portions of an osmotic tablet that comprise an active pharmaceutical ingredient. Drug layers may be solid or liquid. Discussion of solid drug layers may be found in U.S. Pat. Nos. 5,633,011; 5,190,765; 5,252,338; 5,620,705; 4,931,285; 5,006,346; 5,024,842; and 5,160,743, among other places. Discussion of liquid drug layers may be found in U.S. Pat. Nos. 6,419,952; 6,174,547; 6,551,613; 5,324,280; 4,111,201; and 6,174,547, among other places.

“Drug layer end” means, for embodiments of the present invention having a drug layer, the portion of an osmotic tablet that includes the drug layer.

“Push layer end” means, for embodiments of the present invention having a push layer, the portion of an osmotic tablet that includes the push layer.

“Near-infrared spectroscopy system” means a spectroscopy system, capable of providing near-infrared spectra of various materials, preferably osmotic tablets in an embodiment of the invention. Such near-infrared spectroscopy systems are typically coupled, preferably mechanically and/or electronically, to an osmotic tablet handling system. Such coupling facilitates the detection of differences between near-infrared spectroscopic characteristics of the drug layer and the push layer of an osmotic tablet being handled by the osmotic tablet handling system. In an embodiment, the near-infrared spectroscopy system is configured to detect differences between near-infrared spectroscopic characteristics of the drug layer and the push layer of an osmotic tablet being handled by the osmotic tablet handling system.

“Near-infrared spectroscopic characteristics of the drug layer and the push layer of an osmotic tablet” refer to absorbances and/or transmittances and/or reflectances, measured in the near-infrared spectrum, that are characteristic of the drug layer and the push layer.

“Laser drill control system” means a control system that operates to control the drilling actions of one or more laser drills. Such laser drill control systems are typically coupled, preferably mechanically and/or electronically, to the near-infrared spectroscopy system and the osmotic tablet laser drilling system. In an embodiment, the laser drill control system is configured to cause the osmotic tablet laser drilling system to drill at least one orifice in a drug layer end of the osmotic tablet, based on the differences detected by the near-infrared spectroscopy system between near-infrared spectroscopic characteristics of the drug layer and the push layer of the osmotic tablet.

III. Orifice Drilling and Osmotic Tablet Handling Systems

The osmotic tablet laser drilling system can be a generally conventional osmotic laser drilling system. Drilling of orifices and equipment for drilling orifices generally are generally disclosed in U.S. Pat. Nos. 3,916,899, by Theeuwes and Higuchi and in U.S. Pat. No. 4,088,864, by Theeuwes, et al. Further descriptions of osmotic tablet laser drilling systems can be found in U.S. Pat. Nos. 5,658,474; and 5,698,119; both to Geerke. Lasers useful in the practice of this invention comprise those available from Lumonics and Coherent. Other lasers may also be useful in the practice of the present invention.

Osmotic tablet handling systems useful in the practice of this invention may be found in U.S. Pat. Nos. 5,658,474; and 5,698,119; both to Geerke. Additionally, osmotic tablet handling systems useful in the practice of this invention can be constructed from pharmaceutical tablet printing systems, such as the Delta™ series of products (available from R W Hartnett, Philadelphia, Pa.), and the VIP™ printer systems (available from Ackley Machine Corporation, Moorestown, N.J.).

Once the location of the drug layer has been established, there are several configurations of the osmotic tablet handling system and the osmotic tablet laser drilling system that can be utilized. In one embodiment, the osmotic tablet handling system can be configured to present an end of an osmotic tablet that has been established as the drug layer end to an osmotic tablet laser drilling system for drilling of at least one orifice. In another embodiment, the osmotic tablet laser drilling system can be configured to be able to drill orifices at different locations on the osmotic tablet, including at one end and a different end of the osmotic tablet. The embodiment might comprise, for instance, multiple laser drills focused on multiple locations on the osmotic tablet. In this embodiment, the osmotic tablet handling system operates to maintain the location and orientation of the osmotic tablet once the near-infrared spectroscopy system and/or the laser drill control system has established the location of the drug layer. At this point, the laser drill control system may operate to activate only those laser drills that are focused on the drug layer end of the osmotic tablet, leaving those laser drills that are focused on the push layer end of the osmotic tablet unactivated.

The laser drill control system is coupled to the near-infrared spectroscopy system and the osmotic tablet laser drilling system. The laser drill control system may comprise hardware, such as computers, an osmotic tablet laser drilling system and software that operates to control the osmotic tablet laser drilling system.

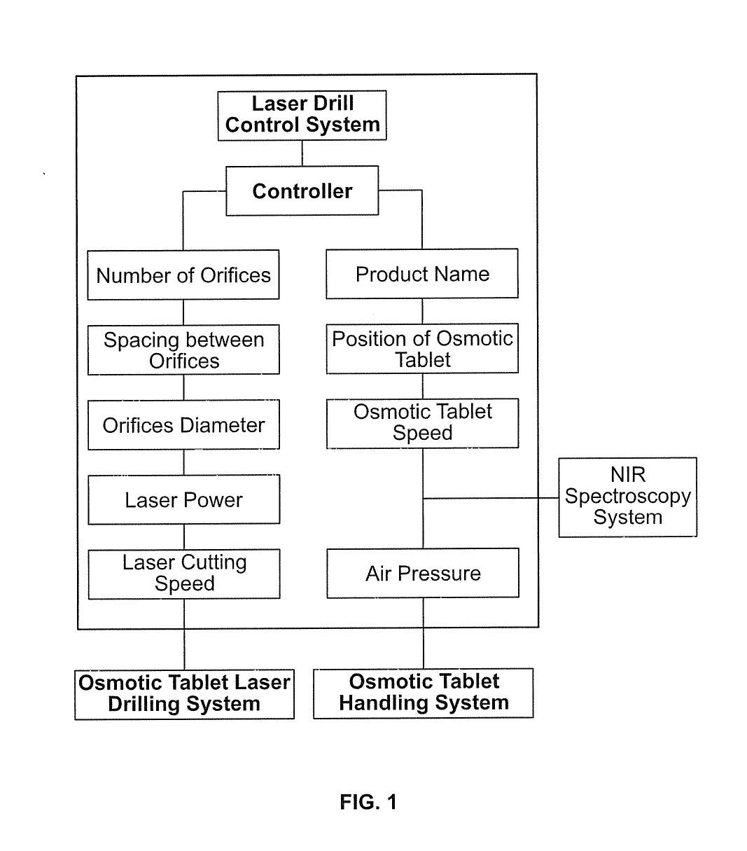

FIG. 1 shows a schematic embodiment of the present invention with emphasis on the inventive laser drill control system. In an embodiment, the laser drill control system takes several user entered parameters, such as number of orifices per osmotic tablet or laser cutting speed, and other information obtained for example from the near-infrared spectroscopy system. The laser drill control system then processes that information, and controls the drilling of orifices by the osmotic tablet laser drilling system. The actual computing resources can be networked so as to be located in a convenient location. Algorithms and programs for the inventive laser drill control system can be conventionally modified to suit a variety of systems or components.

IV. Near Infrared Spectroscopy Systems

A variety of near-infrared spectroscopy systems are useful in the practice of this invention. Generally speaking, such systems should be capable of withstanding manufacturing environments, and capable of non-contact measurement of the osmotic tablets. Examples of equipment useful in the assembly of near-infrared spectroscopy systems include, but are not limited to, the Luminar 4030 Miniature Free Space™ Process NIR Analyzer (available from Brimrose), or the Visio Tec™ line of products (available from Uhlmann Visio-Tec GMBH, Laupheim, Germany).

Various additional equipment besides the NIR analyzer may be needed to construct a near-infrared spectroscopy system according to the invention. Such additional equipment may include, but is not limited to, air flow curtains, mounting hardware, various optical modules, computers, networking hardware, and analytical and operating software. Other hardware or software that might be needed to complete the inventive near-infrared spectroscopy system would be determinable by one of skill in the art.

In an embodiment, the near-infrared spectroscopy system comprises more than one optical module. In a preferred embodiment, the near-infrared spectroscopy system comprises at least one optical module positioned to detect the near-infrared spectroscopic characteristics of one end of the osmotic tablet, and at least one optical module positioned to detect the near-infrared spectroscopic characteristics of a different end of the osmotic tablet. This allows the laser drill control system to establish substantially simultaneously, or even simultaneously, which end is the push layer end and which is the drug layer end of the osmotic tablet. Typically, the optical modules may be placed 0.5 inches or less away from the osmotic tablet being scanned, preferably 0.2 inches or less away from the osmotic tablet being scanned.

Near-infrared spectroscopy systems, and laser drill control systems according to the invention may be configured to detect and register differences between near-infrared spectroscopic characteristics of the drug layer and the push layer. Generally, the performing of the spectroscopic measurements is handled by the near-infrared spectroscopy system. The processing of those measurements to register the detection of differences may be performed wholly by the near-infrared spectroscopy system, wholly by the laser drill control systems, or by the combination of the two systems.

In certain preferable embodiments, the near-infrared spectroscopy system may be configured to scan the osmotic tablets in reflectance mode, although transmission mode also may be useful. An advantage of reflectance mode is that only one optical module per tablet end may be needed in certain embodiments. Preferably, the near-infrared spectroscopy system may be configured to scan wavelengths from about 1100 nm to about 2200 nm. As discussed below, once the near-infrared spectroscopic characteristics of the push layer and the drug layer have been determined for a particular osmotic tablet type, not all wavelengths need to be scanned during operation of the near-infrared spectroscopy system in order to detect differences between near-infrared spectroscopic characteristics of the drug layer and the push layer of an osmotic tablet being handled by the osmotic tablet handling system. Selecting narrower wavelength ranges provides for a faster scan speed, which may result in overall higher throughputs for the inventive apparatus or method.

There are a number of algorithms that can be used to establish differences in near-infrared spectroscopic characteristics of the drug layer and the push layer, once near-infrared spectra of the drug layer and push layer have been determined. In a first method, if there is an obvious difference between the two near-infrared spectra (one for the drug layer and one for the push layer) then a simple difference between values at a particular wavelength, or in a different embodiment more than one wavelength, is enough to establish a differentiation.

In a second method, if there is a less than obvious difference between the two near-infrared spectra, then a region with a change in slope is chosen for evaluation. In Near-infrared Spectra #1, a region with a change in slope is chosen and an integration is performed. The difference between points on the y-axis (y2−y1) is divided by the difference between points on the x-axis (x2−x1). This will result in a slope value assigned to the Near-infrared Spectra #1. The same procedure is done for Near-infrared Spectra #2. The slope values from the two curves are compared. One value will be higher than the other. This will identify one curve with respect to the other curve. As an example, Near-infrared Spectra #1 may have a steep slope between points x2 and x1. In other words, (y2−y1) is a large number. If Near-infrared Spectra #2 is almost flat in the same region, (y2−y1) for the Near-infrared Spectra #2 will be a small number. When comparing the values of Near-infrared Spectra #1 and Near-infrared Spectra #2, one will be large and one will be small. The difference in slope values between the two near-infrared spectra can enable the location of the push layer and the drug layer.

Similarly, regions of the two near-infrared spectra can be surveyed for providing a positive slope value versus a negative slope value. This may lead to even easier identification of one near-infrared spectra versus the other, and thus to easier detection and location of the push and drug layers.

The wavelength ranges chosen for evaluation are selected after viewing several near-infrared spectra for each desired condition (drug layer, push layer, etc). Basically a “library” is created for each desired condition. Once the region is identified, the spectrum scan is narrowed down to only look at the small region previously identified as providing a difference between the two curves. This speeds up the detection of differences between near-infrared spectroscopic characteristics of the drug layer and the push layer. It may also speed up the overall drilling operation, because the less time it takes to locate the drug layer, the less overall time it takes to drill the orifice (in an embodiment, the complete drilling step may include both location and drilling).

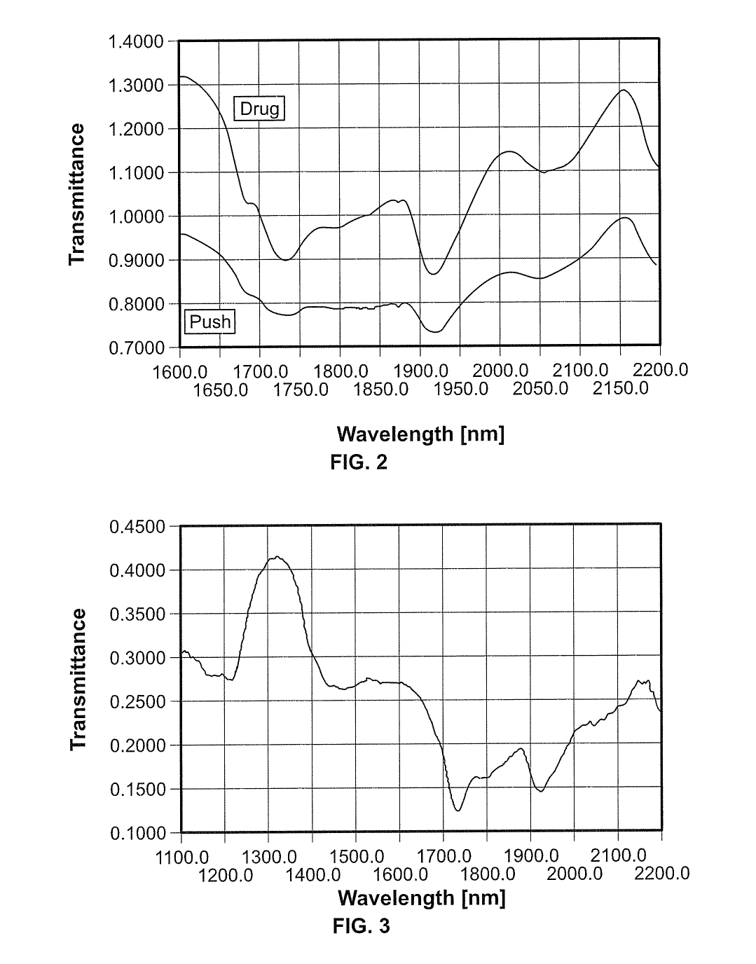

FIG. 2 shows a near-infrared spectrum obtained for the solid drug layer and the push layer of an osmotic tablet. The spectrum responses are significantly different for each side as can be seen by inspection of FIG. 2. The difference in spectrum responses may be used, as is discussed above, to create an signal that eventually will be used to inform the osmotic tablet laser drilling system as to which side of the osmotic tablet to drill in order that the orifice is correctly placed adjacent to the solid drug layer.

It is not necessary to scan the entire spectrum to make a determination. The responses at individual wavelengths or a narrow range of wavelengths may have significantly (i.e. easily detectable) different responses for the solid drug layer versus the push layer. This reduction in collection of data may result in a much more rapid process.

Slope responses may also be used, as discussed above. As an example, as compared to the slope at 1685 nm on the solid drug layer curve in FIG. 2, there is no corresponding slope on the push layer curve at that wavelength. Another area where a difference can be found is at 1825 nm. On the solid drug layer spectrum, there is an upward slope, but on the solid push layer spectrum, the slope is very small (the curve is essentially flat).

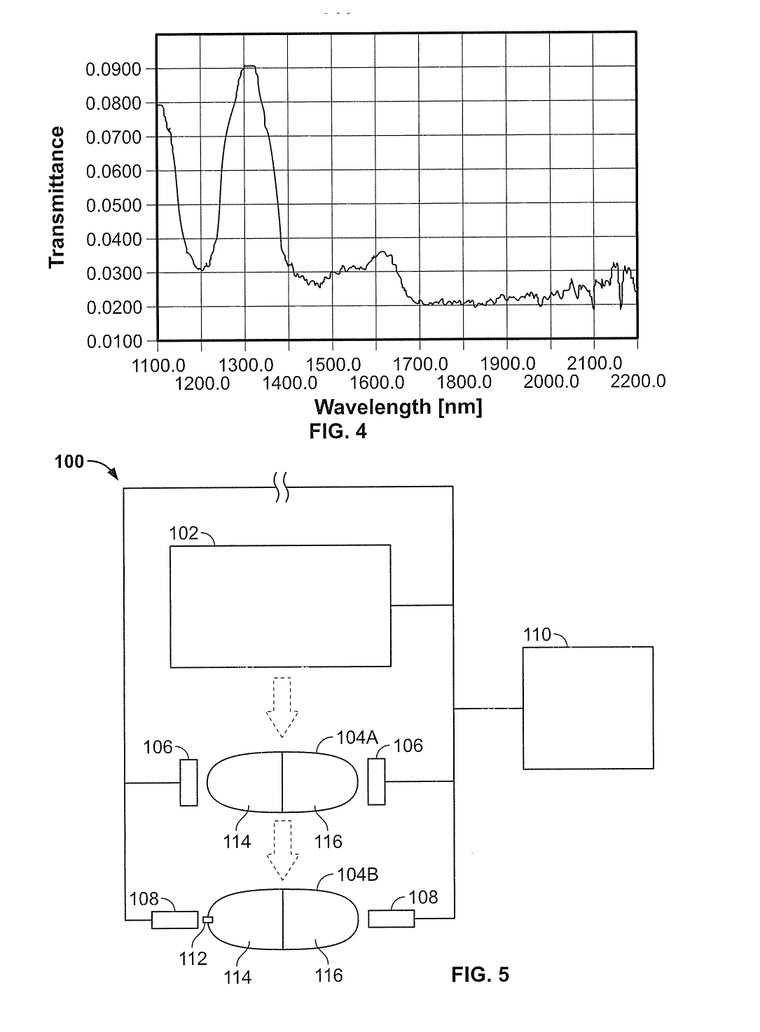

FIG. 3 shows a near-infrared spectrum obtained for a push layer. FIG. 4 shows a near-infrared spectrum obtained for a liquid drug layer. The spectrum responses are noticeably and significantly different. The difference in spectrum responses may be used, as is discussed above, to create an signal that causes the osmotic tablet laser drilling system which side of the osmotic tablet to drill in order that the orifice is correctly placed adjacent to the solid drug layer. It should be noted that the osmotic tablet laser drilling system only partially drills the liquid drug end. The osmotic tablet laser drilling system does not drill the complete orifice through because then the liquid drug layer would leak out of the osmotic tablet. The partially drilled orifice will later create a complete orifice for the liquid drug to exit the osmotic tablet once osmotic tablet is in operation and pressure builds up inside of it.

As noted above, it is not necessary to scan the entire spectrum to make a determination. The responses at individual wavelengths or a narrow range of wavelengths may have significantly (i.e. easily detectable) different responses for the solid drug layer versus the push layer. This reduction in collection of data may result in a much more rapid process. As an example, there is a detectable different in the near-infrared spectroscopic characteristic of the liquid drug layer and the push layer at about 2100 nm. In an embodiment, only this one point of the spectrum might need to be scanned to provide a reliable output. If use of the slope method of differentiation is desired, then in the case of the data shown in FIGS. 3 and 4, attention can be given to the spectra at about 1900 nm. The push layer spectrum exhibits a negative slope at that group of wavelengths. The liquid drug layer spectrum is essentially flat, with a very small slope. The difference in slopes may be enough to create an signal that causes the osmotic tablet laser drilling system which side of the osmotic tablet to drill in order that the orifice is correctly placed adjacent to the liquid drug layer.

V. Embodiments

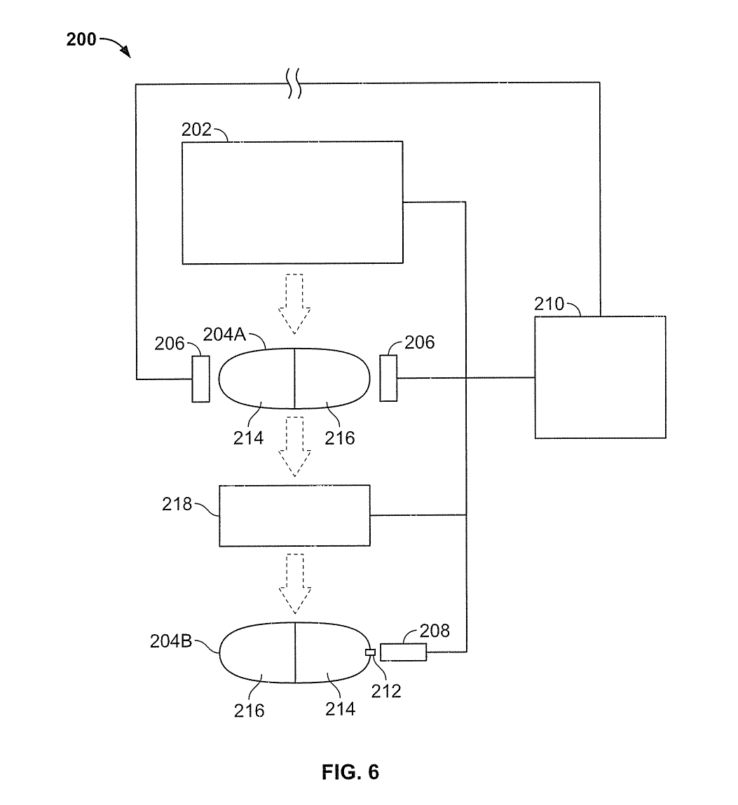

FIG. 5 shows an embodiment of the present invention. Shown is embodiment 100, together with osmotic tablet handling system 102, osmotic tablet in first position 104A, osmotic tablet in second position 104B, near-infrared spectroscopy system 106, osmotic tablet laser drilling system 108, laser drill control system 110, orifice 112, drug layer 114, and push layer 116. Near-infrared spectroscopy system 106 is coupled to osmotic tablet handling system 102, and comprises two optical modules. Optional separate computer components of near-infrared spectroscopy system 106 are not shown. Osmotic tablet laser drilling system 108 is coupled to osmotic tablet handling system 102 and laser drill control system 110. In this embodiment, osmotic tablet laser drilling system 108 comprises two laser drills. In other embodiments, more or less laser drills may be used.

In operation, osmotic tablet handling system 102 functions to move an osmotic tablet into first position 104A. At that point, near-infrared spectroscopy system 106 operates as described above to detect differences between near-infrared spectroscopic characteristics of drug layer 114 and push layer 116. From the signal generated by near-infrared spectroscopy system 106, laser drill control system 110 determines the location of the push layer end of the osmotic tablet. As the osmotic tablet advances to second position 104B, laser drill control system 110 causes osmotic tablet laser drilling system 108 to drill orifice 112 in the drug layer end of osmotic tablet in second position 104B. In an embodiment, osmotic tablet in first position 104A and osmotic tablet in second position 104B are in the same spatial location; for instance osmotic tablet in first position 104A and osmotic tablet in second position 104B may represent the same osmotic tablet. The positions have been shown in different spatial locations in FIG. 5 for ease of illustration only.

FIG. 6 shows an embodiment of the present invention. Shown is embodiment 200, together with osmotic tablet handling system 202, osmotic tablet in first position 204A, osmotic tablet in second position 204B, near-infrared spectroscopy system 206, osmotic tablet laser drilling system 208, laser drill control system 210, orifice 212, drug layer 214, push layer 216, and additional osmotic tablet handling system 218. Near-infrared spectroscopy system 206 is coupled to osmotic tablet handling system 202, and comprises two optical modules. Optional separate computer components of near-infrared spectroscopy system 206 are not shown. Osmotic tablet laser drilling system 208 is coupled to osmotic tablet handling system 202 and laser drill control system 210. In this embodiment, Osmotic tablet laser drilling system 208 comprises one laser drill. In other embodiments, more laser drills may be used. Additional osmotic tablet handling system 218 is coupled to osmotic tablet handling system 202 and to laser drill control system 210.

In operation, osmotic tablet handling system 202 functions to move an osmotic tablet into first position 204A. At that point, near-infrared spectroscopy system 206 operates as described above to detect differences between near-infrared spectroscopic characteristics of drug layer 214 and push layer 216. From the signal generated by near-infrared spectroscopy system 206, laser drill control system 210 determines the location of the push layer end of the osmotic tablet. As the osmotic tablet advances to second position 204B, additional osmotic tablet handling system 218 operates to reorient the osmotic tablet such that its drug layer end is facing osmotic tablet laser drilling system 208. Laser drill control system 210 then causes osmotic tablet laser drilling system 208 to drill orifice 212 in the drug layer end of osmotic tablet in second position 204B. In an embodiment, osmotic tablet in first position 204A and osmotic tablet in second position 204B are in the same spatial location; for instance osmotic tablet in first position 204A and osmotic tablet in second position 204B may represent the same osmotic tablet. The positions have been shown in different spatial locations in FIG. 6 for ease of illustration only.

While there has been described and pointed out features and advantages of the invention, as applied to present embodiments, those skilled in the art will appreciate that various modifications, changes, additions, and omissions in the method described in the specification can be made without departing from the spirit of the invention. The preceding embodiments have been intended to illustrate, and in no way limit, the scope of the present invention.

What is claimed is:

1. An apparatus comprising:
an osmotic tablet handling system for handling osmotic tablets that comprise a drug layer and a push layer;
an osmotic tablet laser drilling system for drilling at least one orifice in a drug layer end of the osmotic tablet;
a near-infrared spectroscopy system; and
a laser drill control system;
wherein the osmotic tablet laser drilling system is coupled to the osmotic tablet handling system and the near-infrared spectroscopy system, and the near-infrared spectroscopy system is coupled to the osmotic tablet handling system and a laser drill control system, and the laser drill control system is coupled to the near-infrared spectroscopy system and the osmotic tablet laser drilling system.

2. The apparatus of claim 1, wherein the drug layer comprises a solid drug layer.

3. The apparatus of claim 1, wherein the drug layer comprises a liquid drug layer.

4. The apparatus of claim 1, wherein the near-infrared spectroscopy system is configured to detect differences between near-infrared spectroscopic characteristics of the drug layer and the push layer.

5. The apparatus of claim 4, wherein the differences between near-infrared spectroscopic characteristics of the drug layer and the push layer comprise different transmittances or absorbances over at least one near-infrared wavelength.

6. The apparatus of claim 4, wherein differences between near-infrared spectroscopic characteristics of the drug layer and the push layer comprise different transmittances or absorbances over more than one near-infrared wavelength.

7. The apparatus of claim 1, wherein the osmotic tablets comprise an opaque coating.

8. The apparatus of claim 1, wherein the near-infrared spectroscopy system comprises at least one optical module.

9. The apparatus of claim 8, wherein at least one optical module is positioned to detect the near-infrared spectroscopic characteristics of one end of the osmotic tablet, and at least one optical module is positioned to detect the near-infrared spectroscopic characteristics of a different end of the osmotic tablet.

10. A method comprising:
handling an osmotic tablet that comprises a drug layer and a push layer;
detecting differences between near-infrared spectroscopic characteristics of the drug layer and the push layer of the osmotic tablet being handled; and
causing an osmotic tablet laser drilling system to laser drill at least one orifice in a drug layer end of the osmotic tablet, based on the differences detected between near-infrared spectroscopic characteristics of the drug layer and the push layer of the osmotic tablet.

11. The method of claim 10, wherein the drug layer comprises a solid drug layer.

12. The method of claim 10, wherein the drug layer comprises a liquid drug layer.

13. The method of claim 10, wherein the differences between near-infrared spectroscopic characteristics of the drug layer and the push layer comprise different transmittances or absorbances over at least one near-infrared wavelength.

14. The method of claim 13, wherein differences between near-infrared spectroscopic characteristics of the drug layer and the push layer comprise different transmittances or absorbances over more than one near-infrared wavelength.

15. The method of claim 10, wherein the osmotic tablet comprises an opaque coating.

16. An apparatus comprising:
means for handling an osmotic tablet that comprises a drug layer and a push layer;
means for detecting differences between near-infrared spectroscopic characteristics of the drug layer and the push layer of the osmotic tablet being handled; and
means for causing an osmotic tablet laser drilling system to laser drill at least one orifice in a drug layer end of the osmotic tablet, based on the differences detected between near-infrared spectroscopic characteristics of the drug layer and the push layer of the osmotic tablet.

17. The apparatus of claim 16, wherein the drug layer comprises a solid drug layer.

18. The apparatus of claim 16, wherein the drug layer comprises a liquid drug layer.

19. The apparatus of claim 16, wherein the differences between near-infrared spectroscopic characteristics of the drug layer and the push layer comprise different transmittances or absorbances over at least one near-infrared wavelength.

20. The apparatus of claim 19, wherein differences between near-infrared spectroscopic characteristics of the drug layer and the push layer comprise different transmittances or absorbances over more than one near-infrared wavelength.

21. The apparatus of claim 16, wherein the osmotic tablet comprises an opaque coating.

22. The apparatus of claim 16, wherein the means for detecting differences between near-infrared spectroscopic characteristics of the drug layer and the push layer of the osmotic tablet being handled comprises a near-infrared spectroscopy system.

23. The apparatus of claim 22, wherein near-infrared spectroscopy system comprises at least one optical module.

24. The apparatus of claim 23, wherein at least one optical module is positioned to detect the near-infrared spectroscopic characteristics of one end of the osmotic tablet, and at least one optical module is positioned to detect the near-infrared spectroscopic characteristics of a different end of the osmotic tablet.
